(19) United States
(12) Patent Application Publication
(10) Pub. No.: US 2011/0087181 A1
(43) Pub. Date: Apr. 14, 2011

(54) External catheter tube

(76) Inventors: Richard Bidwell, Uniondale, PA (US); Tara M. Rands, Brooklyn, NY (US)

(21) Appl. No.: 12/588,353

(22) Filed: Oct. 13, 2009

(51) Int. Cl. A61F 5/44

(52) U.S. Cl. 604/328

(57) ABSTRACT

The external catheter tube is a flexible, coiled collection tube that connects a catheter to a urine collection bag. The collection tube has a coiled portion that can readily extend and contract to relieve tension on the catheter. The assembly includes at least on swivel-snap connector. In one embodiment, one end of the connecting tube is connected to the catheter by a by a straight or linear swivel-snap connector, while the opposite end of the tubing is connected to the drainage bag by and elbow or 90 swivel-snap connector. In another embodiment, one end of the connecting tube is molded integrally with the collection tube, while the opposite end of the tubing is connected to the drainage bag by an elbow or 90 swivel-snap connector. In either embodiment, the connecting tube may be configured with the coiled portion extending horizontally or with the coiled portion hanging in a loop.

### BACKGROUND OF THE INVENTION

1. Field of the Invention

The present invention relates to medical devices for urine drainage, and more particularly to an external catheter tube that provides a coiled, flexible collection tube connecting a catheter and a collection bag by one or more swivel-snap connectors.

2. Description of the Related Art

A urinary drainage system typically comprises a catheter, a collection tube, a collection bag and a drainage system for the collection bag. A urinary drainage catheter is a hollow tubular device to permit drainage of urine by insertion of the tube into the bladder through the urethral tract. Foley catheters are commonly employed urinary drainage catheters that are passed through the urethra and into the bladder. The catheter is held in place by a balloon that can be inflated with sterile water upon insertion into the bladder. Use of a catheter is generally reserved for patients who are undergoing surgery, medically immobile, incontinent, or otherwise incapable of voluntary urination.

Patients who are catheterized and ambulatory present a special set of problems. The catheter must be connected to a collection bag and a drainage system for the collection bag must be provided.

A normal catheter drainage tubing exiting the body is connected to a removable collection bag using a length of rubber tubing. The rubber tubing is usually forced into a radius shaped bend to connect to the collection bag that is strapped to the leg. The normal movement of the leg forward, back and side to side causes pulling, pushing or tugging on the catheter. The catheter tubing exiting the body is fixed and the leg bag is movable so the distance between them is constantly increasing and decreasing when a person moves. This movement causes tugging because the rubber tube is not able to lengthen and contract which creates discomfort and sometimes pain.

Thus, an external catheter tube solving the aforementioned problems is desired.

### SUMMARY OF THE INVENTION

The external catheter tube is a flexible connecting tube assembly that connects a catheter to a urine collection bag. The connecting tube has a coiled portion that can readily extend and contract to relieve tension on the catheter. The assembly includes at least one swivel-snap connector. In one embodiment, one end of the connecting tube is connected to the catheter by a straight or linear swivel-snap connector, while the opposite end of the tubing is connected to the drainage bag by and elbow or 90 swivel-snap connector. In another embodiment, one end of the connecting tube is molded integrally with the connecting tube, while the opposite end of the tubing is connected to the drainage bah by an elbow or 90 swivel-snap connector. In either embodiment, the connecting tube may be configured with the coiled portion extending horizontally or with the coiled portion hanging in a loop.

These and other features of the present invention will become readily apparent upon further review of the following specification and drawings.

### BRIEF DESCRIPTION OF THE DRAWINGS

FIG. 1 is a front view of a first embodiment of an external catheter tube according to the present invention, having swivel-snap connectors at both ends of the connecting tube.

FIG. 2 is a front view of the external catheter tube shown in a configuration permitting the coiled portion of the connection tube to hang in a loop.

FIG. 3 is a front view of a second embodiment 13 of an external catheter tube according to the present invention, having one end of the connecting tube integrally molded with the catheter.

FIG. 4 is a front view of the external catheter tube of FIG. 3, shown in a configuration in which the connecting tube is constrained to extend horizontally.

Similar reference characters denote corresponding features consistently throughout the attached drawings.

### DETAILED DESCRIPTION OF THE PREFERRED EMBODIMENTS

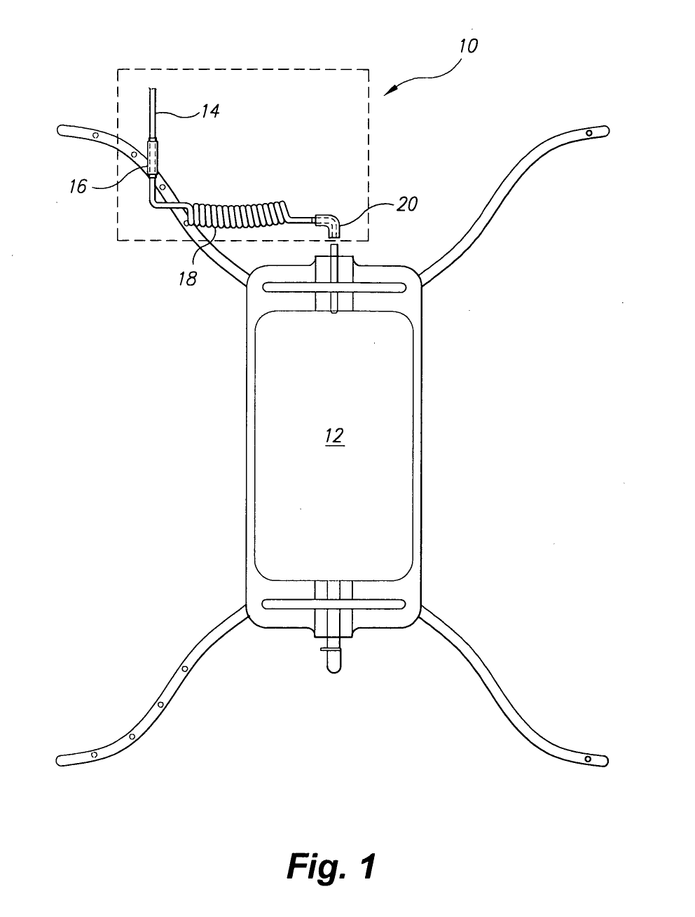

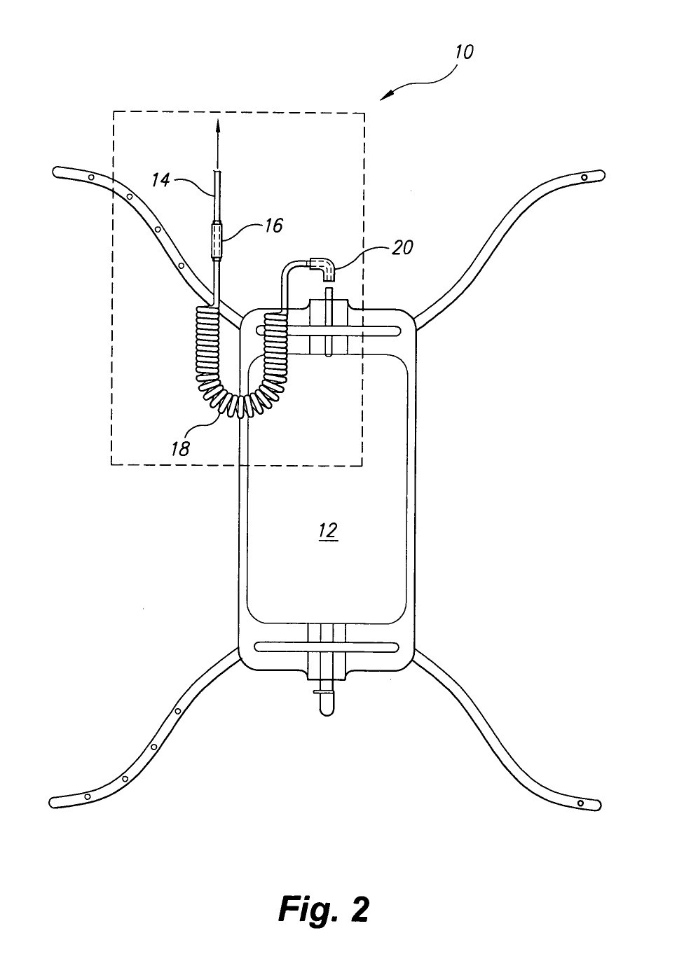

The external catheter tube is a connecting tube assembly that connects a urinary catheter with a drainage or collection bag. FIGS. 1 and 2 show a first embodiment of the external catheter tube designated generally as 10 in the drawings. In this embodiment, the external portion of the catheter 14 is connected to the proximal end of the flexible connecting tube 18 by a straight swivel snap connector 15 that allows the tubing to rotate freely with respect to the catheter 14. The opposite, distal end of the connecting tube 12 is connected to the connecting tube 18 is connected to the collection or drainage bag 12 by and elbow or 90 swivel-snap connector 20 that allows rotation of the flexible tubing with respect to the collection bag 12. The bag 12 is typically is typically strapped to the patient's leg by upper and lower straps. A portion of the connector tube 18 is wound in a helical coil. The coiled portion of the connector tube 18 is sufficiently resilient that the coiled portion may extend and retract with movement of the patients leg to avoid applying tension to the indwelling catheter 14.

FIG. 1 shows the external catheter tube 10 in a configuration in which the coiled portion of the connecting tube 18 is constrained to extend substantially horizontal, which may be achieved by making the connecting tube 18 sufficiently short, or by attaching the collection bag 12 sufficiently low on the patient's leg. FIG. 2 shows the external catheter tube 10 in a configuration in which the coiled portion of the connecting tube 18 is permitted to hang in a loop, which may be achieved by making the connecting tube 18 sufficiently long, or by attaching the collection bag 12 sufficiently high on the patient's leg.

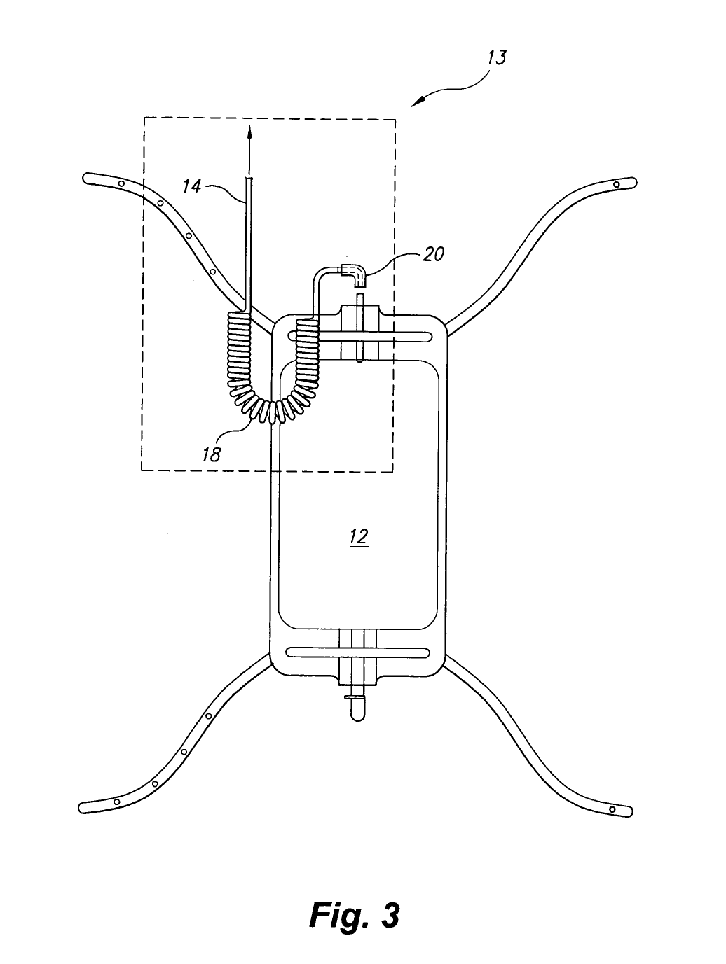

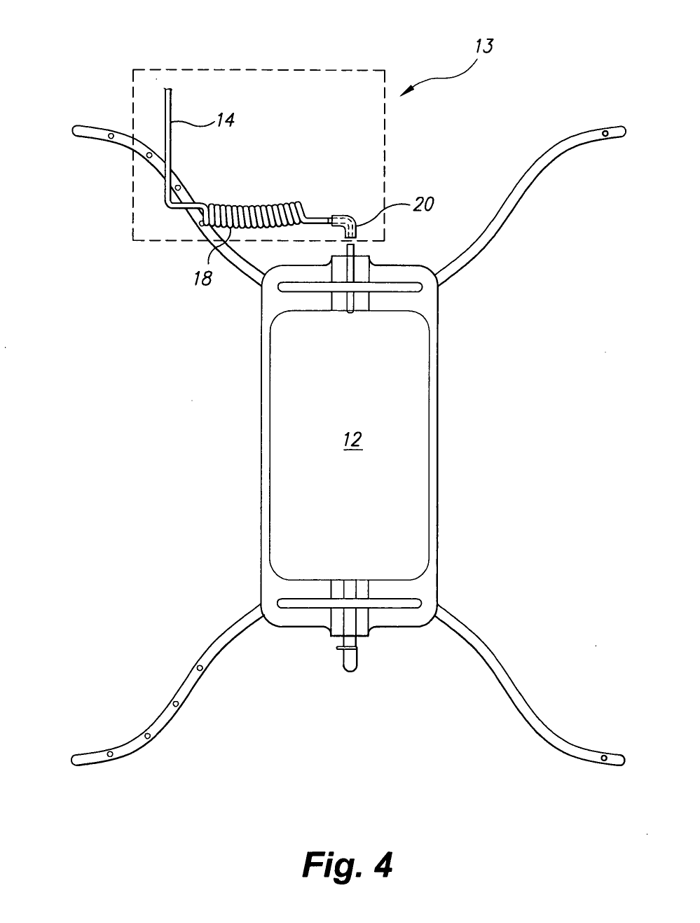

FIGS. 3 and 4 show a second embodiment of the external catheter tube, designated generally as 13 in the drawings. In this embodiment, the external portion of the catheter 14 is formed integrally with the flexible connecting tube 18 by molding, welding, or extrusion during manufacture. The opposite, distal end of the connecting tube 18 is connected to the collection or drainage bag 12 by an elbow or 90 swivel-snap connector 20 that allows rotation of the flexible tubing with respect to the collection bag 12. The bag 12 is typically strapped for the patient's leg by upper and lower straps. A portion of the connector tube 18 is wound in a helical coil. The coiled portion of the connector tube 18 is sufficiently resilient that the coiled portion may extend and retract with the movement of the patient's leg to avoid applying tension to the indwelling catheter 14.

FIG. 4 shows the external catheter 10 in a configuration in which the coiled portion of the connecting tube 18 is constrained to extend substantially horizontal, which may be achieved by making the connecting tube 18 sufficiently short, or by attaching the collection bag 12 sufficiently low on the patient's leg. FIG. 3 shows the external catheter tube 10 in a configuration in which the coiled portion of the connecting tube 18 is permitted to hang in a loop, which may be achieved by making the connecting tube sufficiently long, or by attaching the collection bag 12 sufficiently high on the patient's leg.

Materials suitable for use for the connecting tube of the present invention include polyethylene, polypropylene, polyurethane, vinyl, silastic and other material which may be sterilized, or other tubing materials that are known in the art.

It is to be understood that the present invention is not limited to the embodiments described above, but encompasses any and all embodiments within the scope of the following claims.

We claim:

1. An external catheter tube, comprising:
a coiled, flexible tube having a portion wound into a resilient, helical coil, the tube having a proximal end and a distal end; and
a first connector attached to the distal end of the flexible tube, the first swivel-snap adaptor being adapted for connecting the flexible tube to a urinary bag.

2. The external catheter tube of claim 1, wherein the first swivel-snap connectors is a 90 elbow connector.

3. The external catheter tube of claim 1, further comprising a second swivel-snap connector attached to the proximal end of the flexible tube, the second swivel-snap connector being adapted for connecting the proximal end of the flexible tube to an indwelling catheter.

4. The external catheter tube according to claim 3, wherein said swivel-snap connector comprises a straight connector.

5. The external catheter tube of claim 1, further comprising a urinary catheter integrally formed at the proximal end of the flexible tube.

6. A method of using the external catheter tube of claim 1, comprising the steps of adjusting the step of adjusting the position of the urinary collection bag on a patient's leg so that the coiled section is constrained to extend substantially horizontally.

7. A method of using the external catheter tube of claim 1, comprising the step of adjusting the position of the urinary collection bag on a patient's leg so that the coiled portion of said flexible tube is permitted to hang in a loop.

8. An external catheter tube, comprising:
a flexible, coiled tube having a portion, wound into a resilient, helical coil, the tube having a proximal and a distal end;
a first swivel-snap attached to the distal end of the flexible tube, the first swivel-snap connector being adapted for connecting the flexible tube to a urinary collection bag; and
a second swivel-snap connector attached to the proximal end of the flexible tube, the second swivel-snap connector being adapted for connecting the proximal end of the flexible tube to an indwelling catheter.

9. The external catheter tube according to claim 8, wherein said first swivel-snap connector is a 90 elbow connector.

10. The external catheter tube according to claim 9, wherein said second swivel-snap connector comprises a straight connector.

11. A method of using the external catheter tube of claim 10, comprising the step of adjusting the position of the urinary collection bag on a patient's leg so that the tube is constrained to extend substantially horizontal.

12. A method of using the external catheter tube of claim 10, comprising the steps of adjusting the position of the urinary collection bag on a patient's leg so that the tube is permitted to hang in a loop.

13. An external catheter tube, comprising:
a flexible, coiled tube having a portion wound into a resilient, helical coil, the tube having a proximal and a distal end;
a first swivel-snap attached to the distal end of the flexible tube, the first swivel-snap connector being adapted for connecting the flexible tube to a urinary collection bag; and
a catheter integrally formed at the proximal end of the flexible tube.

14. The external catheter tube of claim 13, wherein said first swivel-snap connector is a 90 elbow connector.

15. A method of using the external catheter tube of claim 14, comprising the step of adjusting the position of the urinary collection bag on a patient's leg so that the coiled portion of said flexible tube is constrained to extend substantially horizontal.

16. A method of using the external catheter tube of claim 14, comprising the step of adjusting the position of the urinary collection bag on a patient's leg so that the coiled portion of said flexible tube is permitted to hang in a loop.
